(19) United States
(12) Patent Application Publication
(10) Pub. No.: US 2021/0277342 A1
(43) Pub. Date: Sep. 9, 2021

(54) Modular Biogas Facility, Method For Operating A Modular Biogas Facility And System For Monitoring And Control

(71) Applicant: Goffin Energy GmbH, Cham (DE)

(72) Inventors: Gregor URBAN, Cham (DE); Thomas SCHMIDT, Bergheim (DE); Martin SCHMIDT, Hoechenschwand (DE); Thomas BRUESE, Langenfeld (DE); Walter DANNER, Simbach (DE)

(21) Appl. No.: 17/186,899

(22) Filed: Feb. 26, 2021

Related U.S. Application Data
(63) Continuation of application No. PCT/IB2019/056790, filed on Aug. 9, 2019.

(30) Foreign Application Priority Data
Aug. 29, 2018 (DE) 102018121050.7

(51) Int. Cl. C12M 1/107; C12M 3/00; C12M 1/00; C12M 1/34; C12M 1/36

(52) U.S. Cl. CPC C12M 21/04; C12M 23/36; C12M 23/44; C12M 23/58; C12M 41/48; C12M 41/12; C12M 41/34; C12M 41/40; C12M 23/40

(57) ABSTRACT

The invention relates to a modular, mobile, compact, multi-stage and highly efficient biogas facility, a method for operating a modular biogas facility, and a system for the computer-assisted, decentralized monitoring and control of at least one modular biogas facility. The system can be equipped with modular, local intelligence and a local control unit. The modular biogas facility is provided with a plurality of tanks for accommodating biomass. The tanks can be fluidically connected to one another. Furthermore, at least one gas reservoir is provided for the biogas produced in the modular biogas facility. Each of the tanks is a module in the biogas facility. Each tank can be positioned in a rigid and cuboidal frame, with the cuboidal frame having six side faces. The side faces of the cuboidal frame define an envelope for the tank.

### CROSS-REFERENCE TO RELATED APPLICATIONS

The present application is filed under 35 U.S.C. §§ 111(a) and 365(c) as a continuation of International Patent Application No. PCT/IB2019/056790, filed on Aug. 9, 2019, which application claims priority from German Patent Application No. DE 10 2018 121 050.7, filed on Aug. 29, 2018, which applications are incorporated herein by reference in their entireties.

### FIELD OF THE INVENTION

The present invention relates to a modular biogas plant. The modular biogas plant includes a plurality of tanks for holding biomass. The plurality of tanks are connected to one another in a fluid manner. Furthermore, at least one gas reservoir is provided which is designed to hold the biogas generated in the biogas plant.

Additionally, the invention relates to a method for operating a modular biogas plant. The power range, in kWh, of the modular biogas plant essentially depends on the digestibility of the biomass fed and the parameters at which the modular biogas plant is operated. In particular, depending on the configuration, a modular biogas plant can be operated in a power range of up to 500 kWh. It goes without saying for a person skilled in the art that the power range can also be extended beyond 500 kWh. The modular biogas plant comprises a plurality of tanks that are configured to hold digestible biomass. From the plurality of tanks, at least two tanks are configured as hydrolysis tanks. At least one further tank of the plurality of tanks is a fermenter tank. Furthermore, at least one reservoir is provided which is suitable for receiving the biogas generated by the biogas plant.

The invention also relates to a system for computer-aided, centralized monitoring and control.

### BACKGROUND OF THE INVENTION

The German patent application DE 10 2008 015 609 A1 discloses a biogas plant and a method for generating biogas. Disclosed here is a method for producing biogas, in particular methane gas, in a multi-stage process. The multi-stage process includes a hydrolysis process and a methane formation process. The hydrolysis process is spatially separated from the methane formation process. The biogas plant itself has at least two hydrolysis tanks and a fermentation tank or fermenter for a methane formation process. The at least two hydrolysis tanks are spatially separated from the downstream fermenter. A disadvantage of the described biogas plant is that it is not modular, not mobile and is not constructed in a compact manner. In addition, this biogas plant does not allow for remote maintenance or remote control. A move of the biogas plant to another location or a different construction on site is not possible.

The German utility model DE 20 2013 101 554 U1 discloses a receptacle of a biogas plant. The receptacle has a bottom and a peripheral wall. The wall of the receptacle is supported on the outside against at least one container. The at least one container holds a technical device that is required to operate the biogas plant.

The Chinese patent application CN 10 41 40 928 discloses a container for a modular biogas plant. The tank body is arranged in the container. A support frame is arranged between the tank body and the container. The outer part of the tank body is surrounded by a heating loop. The heating loop is a water pipe.

In the Chinese patent application CN 10 62 81 996 a modular bioreactor is disclosed. The modular bioreactor includes several facilities required for the digestion or feeding of the biomass. A modular biogas plant, as described by the present invention is not disclosed.

The German patent application DE 199 58 142 A1 discloses a modular biogas plant. The transportable, modular biogas plant comprises a fermenter and an energy section, both of which are separate components. These components are housed in standard transport containers or in standard transport container frames. The fermenter has a rigid shell. The biogas plant described here is a single-stage and non-thermophilic process.

The German patent DE 10 2004 053 615 B3 discloses a method of degradation of biogenic material (dry substrate fermentation). For this purpose, a percolator is equipped with biogenic material. A percolation liquid is separated by a sieve and sprayed back onto the biogenic material. The excess percolation fluid enters a buffer and is from there into the biogas reactor and fermented to biogas. The cleaned percolation liquid is transferred as wastewater to a storage buffer and from there returned to the percolator. A liquid substrate fermentation (the substrate is diluted to be pumpable) is not intended.

The German patent application DE 10 2013 107 621 A1 discloses a central, modular pumping and shredding unit. The modular pump and shredding unit is part of a biogas plant, which has several reaction and/or storage containers that are connected to one another via fluid pipes, as well as conveying devices or pumps. The pump and shredding unit can also be installed in a receptacle, for example in a container or the like.

The German utility model DE 20 2005 012 340 U1 discloses a biogas plant and a module for a biogas plant. The biogas plant includes at least one fermenter and modules that accommodate technical parts of the plant. Technical parts of the system are in particular the control and control technology, pump technology and at least one block-type thermal power station. The modules as an enclosure or housing and the receptacles, receiving the technical units, are prefabricated garages, which are designed accordingly to accommodate the modules. The individual modules are not intended to be stackable.

The German utility model DE 20 2010 000 437 U1 discloses a transportable, modular biogas plant with a connected fermenter space. The fermenter space is formed of at least two, on their front sides connectable or connected with each other, transportable fermenter modules. Each fermenter modules is formed with a substantially flat base area. The fermenter modules have an essentially rectangular cross section.

The German patent application DE 10 2004 062 993 A1 discloses a biogas plant with at least one fixedly mounted fermenter and at least one mobile container unit. The container unit comprises at least two rooms separated from one another by a wall. A technical unit is installed in the space of the container unit adjacent to the fermenter. A device for the use of biogas is mounted in a room facing away from the fermenter. No mobile biogas plant is disclosed here, only a mobility of the technical unit is provided, which is housed in a container unit.

In the prior art, a distinction is essentially made between a multi-stage biogas plant and a conventional, single-stage biogas plant. All of these types of biogas plants are permanently installed at the place of their construction and can therefore no longer be moved. Only individual parts, such as technical units, can be accommodated in containers and are therefore easy to transport. The advantage of a multi-stage biogas plant is that it is up to 30% more effective than a conventional, single-stage biogas plant. On top of that the multi-stage biogas plants can be fed with all types of digestible biomass. The digestibility of the biomass or the organic substances that can be fed into a biogas plant essentially depends on the microorganisms that are present in the biogas plant. In return, the single-stage biogas plants require a simplified, standardized substrate, such as corn or grass silage with constant nutritional values for the microorganisms. The digestion of biomass waste, such as fibrous and cellulose-containing substances is difficult, if not impossible, in single-stage biogas plants.

### SUMMARY OF THE INVENTION

The invention is based on the object of creating a modular biogas plant that is easy to set up, easy to transport, expandable, set up operational at a different installation location at any time and can be designed cost-effectively.

This object is achieved by a modular biogas plant which has a plurality of modules. A plurality of tanks, for receiving biomass, define a portion of the plurality of modules, The tanks of the modular biogas plant comprise at least two hydrolysis tanks and at least one fermenter tank and being fluidly connected to one another. At least one gas storage for the biogas generated in the at least one fermenter tank of the modular biogas plant is provided as well. A plurality of positioning elements is at least provided to the tanks, wherein the positioning elements define six side surfaces which form an envelope for each tank.

Another object of the invention is to create a method for operating a modular biogas plant which can process a large number of different types of biomass and thereby has a higher efficiency in the production of biogas and in the breakdown of organic substances.

The method utilizes a modular biogas plant which comprise at least a plurality of tanks for receiving biomass. At least two of the tanks are configured as hydrolysis tanks and at least one tank is configured as a fermenter tank. At least one gas storage for the biogas generated in the modular biogas plant is provided as well.

The method comprises the steps:

batch-wise filling of the hydrolysis tanks with biomass, wherein a first temperature range and a first pH range are predominating in the hydrolysis tanks;

transferring the biomass from the at least one hydrolysis tank to the at least one fermenter tank by means of a pump;

producing in the at least one fermenter tank biogas from the biomass, transferred from the at least one hydrolysis tank to the at least one fermenter tank, wherein the formation takes place at a second temperature range and a second pH range in the at least one fermenter tank; and

monitoring continuously a production rate of the biogas and if the production rate of the biogas in the at least one of the fermenter tanks falls below a predefined value, biomass is supplied from one of the hydrolysis tanks until the production rate is again above the predefined value.

Another object of the invention is to create a system for computer-aided, centralized monitoring and control of at least one modular biogas plant, which enables largely automated monitoring and control of the plurality of installed modular biogas plants and thereby optimizes the operation of each the modular biogas plants in order to optimize the efficiency of the generation of biogas in the individual modular biogas plants.

This object is achieved by a system for computer-aided, centralized monitoring and control. The system comprises several modular biogas plants, wherein each modular biogas plant has at least two hydrolysis tanks, at least one fermenter tank, at least one pressure less gas storage tank and several housings. At least one data acquisition unit is assigned to several individual and movable modules of each of the modular biogas plants. Each of the modules has at least one actuator and/or at least one sensor and/or at least one measuring point, which are communicatively connected to the least one data acquisition unit. A communication device which is assigned to each of the modular biogas plants and delivers data from the data acquisition unit to a cloud or receives data from the cloud. A central control and monitoring unit is communicatively connected to the cloud in order to monitor the modular biogas plants in a centralized manner and to control them automatically. A user interface is assigned to each of the modular biogas plants to which messages or warnings can be transmitted from the central control and monitoring unit.

The modular biogas plant is characterized by a plurality of tanks that are designed to hold biomass. The large number of tanks can be fluidly connected to one another, so that biomass can be exchanged or pumped between the individual tanks. The biomass can be freely distributed between the tanks. The individual tanks can also be used as required. For example, tanks for hydrolysis can be used as fermentation tanks and vice versa. Furthermore, at least one gas storage reservoir is provided, which is suitable for accepting the biogas generated by the modular biogas plant. Each of the tanks of the modular biogas plant forms a module of the biogas plant. Several positioning elements are provided per tank. The positioning elements are attached to the tank in such a way that they define a cuboid frame. The cuboid frame defines six side surfaces that form an envelope for the tank. Preferred, the installation of the tanks of the modular biogas plant at the installation site is essentially the same, which results in a significant cost savings and a reduction in the number of parts. For the fixation of the tanks or modules of the modular biogas plant, several anchoring elements can be provided in the floor of the installation site, which with the positioning elements cooperate. This means that the tanks and modules are safely positioned at the site of the biogas plant. The modules of the modular biogas plant can also be easily picked up and relocated to another site. The mobile biogas plants can also be easily expanded or dismantled. The expansion or dismantling is based on the requirements placed of the mobile biogas plants. For example, modules for disinfection, separation (separation of solid and liquid components of the fermented biomass) and drying of the separated, solid components of the fermented biomass can be added.

The modular and variable set-up of the biogas plants saves resources, because once a biogas plant has been set up, it can be used at a different installation site at any time without any major construction work or it can be expanded, if required, or dismantled. Another advantage of the modular biogas plant is that it is mobile, compact, can run a multi-stage process and is highly efficient in the utilization of the biomass applied.

According to a further and advantageous embodiment, there are several positioning elements attached to a rigid cuboid frame. Here the six side surfaces of the rigid, cuboid frame define the envelope for the tank.

The envelope, which is formed by the positioning elements and the rigid, cuboid frame respectively, has the advantage that the possible connection elements or attachments for the tank are located within the envelope. This reduces or prevents damage to the connection elements or attachments during the transport of the tanks.

In one embodiment, the tanks themselves are made of a rigid and dimensionally stable material. According to the possible embodiment, the rigid and dimensionally stable tank can, as already mentioned above, preferably be surrounded by the rigid and cuboid frame. As material for the tanks an acid-resistant plastic, glass fiber reinforced plastic, stainless steel, wood or a laminate made of different materials are conceivable. At least the layer of the tank facing the biomass must be acid-resistant and alkali-resistant.

In another embodiment, the tank can be made from a flexible material. The tank is also positioned in the rigid frame, wherein the side surfaces of the rigid frame are preferably provided with a rigid, dimensionally stable covering, so that the filled tank remains within the outline of the module. According to this embodiment, the connections required for the tank are provided in the dimensionally stable covering at the rear end of the tank and/or front end of the tank.

In another embodiment, the modules of the modular biogas plant further comprise at least two lockable housings. Each of these lockable housings has the size of the cuboid frame. Each housing has a door or an access opening formed on at least one side surface. The remaining side surfaces of the frame have a cladding. Here, too, it is advantageous that the modules for the housings correspond in size to the modules for the tanks. This makes the transport of the elements of the modular biogas plant much easier and standardized, respectively.

According to a first embodiment of the modular biogas plant, a first housing contains a combined heat and power plant that uses the biogas generated in the modular biogas plant as an energy source. A second enclosure contains a control electronic for the entire modular biogas plant, at least one pump, at least one heating device, and a compressed air control for the generation and distribution of compressed air. The second housing preferably has a partition to separate the control electronics for the modular biogas plant from the pump and the heating device. A control room with visualization can also be formed in the room for the control electronics. The pump is used for the controlled transport of the biomass within the biogas plant. The heating device is used for the controlled temperature setting of the tanks of the modular biogas plant. The heating device can be fluidly connected to the corresponding tanks as required in order to set the temperature in the selected tank or tanks.

According to a second embodiment of the housing designed as a module, a first housing contains a combined heat and power plant which uses the biogas generated in the modular biogas plant as an energy source. A second housing is used to accommodate control electronics for the entire modular biogas plant. The second enclosure can be divided by a partition wall in a room with the control electronics and a control room with a visualization of the processes in the modular biogas plant. A third housing includes at least one pump that transports the biomass within the modular biogas plant between the individual tanks. Likewise, a heating device can also be provided in the third housing, which can be used for the controlled temperature setting of the tanks. Furthermore, a compressed air control can also be provided in the third housing.

According to a further embodiment, the at least one pump is connected to the tanks of the modular biogas plant via a pipe system. A controllable and adjustable valve is assigned to each of the tanks. With the controllable and adjustable valve, the biomass can thus be moved between different tanks. The control electronics or a central control and monitoring unit regulates the actuation of the corresponding valves so that the entire modular biogas plant works as effectively as possible and generates biogas. The heating device also has a pipe system that leads to the tanks. Here, too, a controllable and regulatable valve is assigned to each tank in order to enable the individual setting of a required temperature range of the biomass in the individual tanks.

According to a further embodiment, the tanks of the modular biogas plant comprise hydrolysis tanks and fermentation tanks. In this case, at least each of the tanks has at least one connection for a supply of biogas and a connection for a discharge of biogas. It goes without saying for a person skilled in the art that other combinations of connections and also the number of connections can vary. The configuration of the tanks described above is used for description purposes of the invention only and should not be interpreted as a restriction of the invention.

According to one embodiment, the inventive modular biogas plant has at least two hydrolysis tanks. Preferred, the hydrolysis tanks are filled using the BATCH process. For example, the first hydrolysis tank is first filled, and the hydrolysis process is started at a temperature which is selected from a first temperature range. The first temperature range preferably extends from 40° C. to 65° C. The pH range of the hydrolysis can be in the range from 2 to 9. After the first hydrolysis tank has been filled, the second hydrolysis tank is also filled using the BATCH process, so that the hydrolysis can also start there. Depending on the control, finished “hydrolyzate” (hydrolyzed biomass) is pumped or transferred from the first hydrolysis tank (the previously filled hydrolysis tank) into the fermenter tanks. The modular biogas plant according to the invention has the advantage that pump paths can be executed in a controlled manner. It can therefore be pumped to and from any tank of the modular biogas plant. The transfer to the fermenter tank is controlled, so that the rate biogas generation in the fermenter tank always remains substantially constant. By perfecting the biochemical process in the multi-stage biogas plant, about 99.5% or more of the possible biogas can be obtained from one ton of biomass used. Every single tank can be individually and differently temperature controlled. This has the advantage that the conditions for the hydrolysis and/or the fermentation in the tanks can be set and adjusted differently.

According to a further possible embodiment, the modular biogas plant can also be assigned a fermentation residue storage. The fermentation residue storage is used to collect the fermented biomass residues from the fermenter tank or the fermenter tanks of the modular biogas plant. Where applicable, a secondary fermentation can take place in the fermentation residue storage, so that biogas produced in the fermentation residue storage can also be used for further purposes. Generally, the biogas plant according to the invention does not need the fermentation residue storage, since the biogas plant according to the invention ensures an essentially complete fermentation of the biomass in the fermenter tanks. In the event that a fermentation residue storage facility is planned, it will be possible to transfer the fermented biomass residues from the fermenter tanks to the fermentation residue storage. It is therefore necessary that the fermentation residue storage is fluidly connected with the fermenter tanks. The fermentation residue or the fermentation residue storages can also be designed as transportable modules, which have the form of tanks. According to another embodiment, the fermentation residue storage can also be permanently installed at the installation site of the modular biogas plant.

According to a further possible embodiment of the invention, the modular biogas plant can be provided with a pressure less gas storage. In biogas plants, the biogas is usually stored in pressure less or low-pressure storage tanks in a range of 0.05 to 50 mbar overpressure. The pressure less gas storage of the biogas plant according to the invention is formed by a movable foil membrane that meets the corresponding safety requirements for gas storage. The pressure less gas storage tank is used to take in biogas from the modular biogas plant and if necessary, for taking in biogas from an existing fermentation residue storage. Additionally, the pressure less gas storage tank is used for the delivery of biogas to the block-type thermal power plant and for returning biogas to the tanks. To achieve this, an appropriate gas pipe system is required.

According to an advantageous embodiment of the invention, the pressure less gas storage is made of a flexible material and for the transport of the individual modules it is housed in a transport housing with a cladding. After the module with the flexible pressure less gas storage tank has been installed, it can be rolled out at the installation site. To do this, the module (transport housing) is opened accordingly. At one end, the gas storage tank is still connected to a cladding of the transport housing. The cladding of the transport housing has appropriate connections, so that a simple and quick connection of the gas system to the pressure less gas storage can be achieved at the installation site.

According to one embodiment and due to legal regulations in Germany, there is the requirement that the fermentation residue storage needs to be connected to the pressure less gas storage for supplying biogas from the fermentation residue storage to the pressure less gas storage, if any. This has the advantage that further fermentation of the biomass transferred from the fermenter tanks to fermentation residue storage, may take place in the fermentation residue storage and additional biogas generated in the fermentation residue storage. Another use of the biogas generated in the fermentation residue storage is possible.

The high modularity of the inventive modular biogas plant has the advantage that it can be set up quickly at the installation site, since a large part or at least most of the elements of the modular biogas plant are already prefabricated and “ready to use”. The pipes required to connect the individual modules of the biogas plant are also delivered in a module at the installation site. Thus all or at least most of the pipe connections between the individual modules are prefabricated, so that the assembly of the modular biogas system at the installation site can be carried out quickly and in a defined manner. Another advantage is that all modules are of the same size, which ultimately makes transport and logistics much easier. The reduction in the variety of parts and the standardization thus lead to a reduction in costs in the production of the biogas plant. The possibility of the flexible arrangement and the stackability of the modules of the modular biogas plant lead to q reduced footprint and an optimized use of space.

The inventive method is used to operate a modular biogas plant. In particular, the modular biogas plants are configured for a power range up to 500 kWh. It goes without saying for a person skilled in the art that the performance range specified should not be considered as a limitation of the invention. Most of the installed biogas plants will preferably have an output range of 10 to 500 kWh. Depending on customer requirements, power ranges above 500 kWh can also be implemented. The modular biogas plant comprises at least a plurality of tanks for receiving biomass. There are at least two tanks which represent hydrolysis tanks. At least one other tank is a fermenter tank. Furthermore, at least one gas storage tank is provided for the biogas generated by the modular biogas plant. First, the hydrolysis tanks are filled batch-wise with biomass. A temperature from a first temperature range is set in the hydrolysis tanks. During hydrolysis, the pH value is in the Hydrolysis tanks within a pre-defined pH range. Filling the hydrolysis tanks batch-wise means that a tank is almost filled completely, depending on a specified time interval. For example, the predetermined time interval can be one day, so that the same hydrolysis tank is filled every second day. The biomass for filling the hydrolysis tanks can include, for example and without limiting the invention, chicken manure, duck manure, grass silage, corn silage, straw, food waste, slaughterhouse waste, and much more. In principle fats, proteins and carbohydrates can be used as biomass in the modular biogas plant. In general, the inventive biogas plant can process or digest everything that contains fats, oils, fatty acids, lipids, oil-like substances, proteins, starches, sugar, cellulose, hemicellulose, chitin and similar hydrocarbons. Hydrolysis and acidification take place in the hydrolysis tank. The hydrolysis and the acidification both take place in the first temperature range and in the first pH range. During hydrolysis, fatty acids, amino acids and alcohols are built up. During acidification, volatile fatty acids and alcohols are built up.

In a further step of the process according to the invention, biogas is produced in at least one fermenter tank, wherein the biomass is transferred from the hydrolysis tanks to the at least one fermenter tank. The production of biogas takes place in a second temperature range, such as from 35 to 60° C. and at a second pH range, such as from 6.5 to 8.5, instead. The fermentation in the fermenter tanks is also divided into acetic acid formation (acidification) and methanation. During acetification, Acetic acid, carbon dioxide and hydrogen are build up. During methanation, methane and carbon dioxide are built up, wherein methane for example having a proportion of 55% to 75%. It goes without saying for a person skilled in the art that the methane content mentioned above is not intended to constitute a limitation of the invention.

In order to keep the production process of biogas of the modular biogas plant effective, the production rate of the biogas of the modular biogas plant is continuously monitored. In the event that in one of the fermenter tanks the production rate falls below a predefined value, biomass is supplied from one of the hydrolysis tanks until the production rate is above the predefined value again. This has the advantage that the production of biogas of the modular biogas plant takes place effectively, so that the yield of the biogas from the modular biogas plant is always at a high level and maximum organic degradation of the fed biomass takes place.

In one embodiment, at least controllable valves are assigned to the hydrolysis tanks and the fermenter tanks. The hydrolysis tanks and the fermenter tanks are connected via pipes. At least one pump is provide so that the hydrolysis tanks and/or the fermenter tanks can be connected in any combination and that biomass the hydrolysis tanks and/or the fermenter tanks can optionally be supplied or optionally discharged from form the respective tanks.

According to one embodiment, the hydrolysis tanks and the fermenter tanks are each provided with an inlet and outlet for heating fluid. A controllable valve is provided for each inlet and outlet, so that the heating fluid can be fed to the hydrolysis tanks and/or the fermenter tanks in a controlled manner with at least one heating fluid pump. It can thereby be achieved in each case that the temperature in the hydrolysis tanks or fermenter tanks is kept in the temperature range required for the hydrolysis or fermentation at the required temperature level.

According to a further embodiment of the method according to the invention, at least one fermentation residue storage is provided. This at least one fermentation residue storage has also a controllable valve in a pipe to the least one fermentation residue storage. With the controllable valve it is thus possible that controlled from at least one of the fermenter tanks biomass can be fed for a possible secondary fermentation to the at least one fermentation residue storage. The at least one fermentation residue storage facility can also be provided with a gas pipe to the gas storage tank in order to remove any biogas, that may arise from the fermentation residue storage, to the gas storage tank.

According to a further embodiment, at least biogas from the at least one fermenter tank is fed to the gas storage tank without pressure or at low pressure.

According to a further embodiment, the biogas is taken from the gas storage tank in a controlled manner and fed it to a block-type thermal power plat to generate energy from the biogas provided by a module of the biogas plant. It is also possible to compress the biogas from the gas storage tank with a compressor so that the compressed biogas can be blown in a at least controlled manner into a fermenter tank filled with the biomass. By blowing biogas into the biomass of the fermenter tanks mix the biomass and thus improve the production of biogas, which leads to an improvement in the quality of the biogas (possible increase in the CH₄ content and reduction in the CO₂ and H₂S content).

With the inventive method and its application in the modular biogas plant, a large number of organic waste or energy crops can be processed. As energy plants can be processed with the inventive biogas plant, for example, but without limiting the invention, corn or corn silage, Sudan grass, corn on the cob, grass, clover, rye, sugar beet, wheat, potatoes, etc. In the event that the modular biogas plant also has a module for separating and a module for drying the fermented biomass, the dried biomass can, for example, be used to obtain raw materials from the fermented residues of the biomass. Likewise, with the inventive modular biogas plant, agricultural organic waste, such as, but without limiting the invention, liquid cattle manure, liquid pig manure, duck manure layers, bird manure, horse manure, cereal products, straw, waste from the olive harvest (olive pits), vinasse, sugar cane, corn stalks, etc. are processed. Likewise, with the modular biogas plant according to the invention, industrial biowaste (industrial by-products, organic waste and biowaste), such as, but without limiting the invention, an overproduction of beer, bread, muesli bars, vegetables, fruits, as well as animal meal, blood (slaughterhouse waste), carcass meal, fruit waste, Chinese cabbage, whey, ice cream, milk waste, etc. are processed. In addition, any other organic waste or biowaste, such as that generated in kitchens or restaurants, can be processed with the modular biogas plant.

The invention also enables a system for computer-aided, centralized monitoring and control of at least one biogas plant. The inventive system according to the computer-aided, centralized monitoring and control can advantageously be applied to biogas plants in a wide range of performance. The system comprises several modular biogas plants, whereby each of the modular biogas plants comprise several individual movable modules. At least one actuator and/or one sensor and/or one measuring point is assigned to each of the modules. The actuators and/or sensors and/or measuring points are communicatively connected to at least one data acquisition unit. Furthermore, a communication device is assigned to each of the modular biogas plants. Via the communication device, the data of the data acquisition unit can be delivered to a cloud or data or signals can be received from the cloud and used for control of the modular biogas plants by the data acquisition unit. A central control and monitoring unit is communicatively connected to the cloud and is used for centralized monitoring and automatic control of the modular biogas plants on site. Furthermore, each of the modular biogas plants has one assigned user interface. The user interface can be remotely controlled from the central control and monitoring unit, and messages or warnings can be sent to the local controller. The warnings or messages are marked colored for the user or the operator of the respective local modular biogas plant in order to alert him to or to point out necessary actions. The messages and warnings sent out by the central control and monitoring unit to enable the user to intervene immediately with the respective modular biogas plant in order to rectify possible errors in advance. This has the advantage that the modular biogas plant concerned can work continuously and downtimes can be largely avoided. In this embodiment, the data acquisition unit is communicatively connected to at least one controller.

Condition-based maintenance (CBM), for example, can also be implemented through the central control and monitoring unit. The CBM functions of remote maintenance and trend analyzes allow the user of the modular biogas plant to be informed of possible errors or failures at an early stage.

According to a further embodiment of the invention, an intelligent head station is assigned to each module of the modular biogas plant. The intelligent head station comprises a data acquisition unit with the communication device. The parameters of the respective module of the modular biogas plant are stored in the intelligent head station. The intelligent head station can therefore also take over the control of the respective module. The intelligent head stations of the modular biogas plant are communicatively connected to each other and to the cloud. The control for the modules is implemented in the cloud.

In one embodiment, the plurality of modules of the modular biogas plants comprise at least two hydrolysis tanks and several fermenter tanks. A pressure less gas storage tank is also provided, which takes up the biogas at least from the fermenter tanks. In addition, several modules are designed as housings in which, for example, but without limiting the invention, corresponding elements for the control electronics and the operation of the modular biogas plants are accommodated.

In one embodiment, a block-type thermal power plant is provided in a first housing, which can be operated via the central control and monitoring unit. With the block-type thermal power plant, the biogas generated, can be used in the modular biogas plants as an energy source.

In one embodiment, control electronics for the modular biogas plants are housed in a second housing. The control electronics are connected to the individual actuators, sensors, etc. or the head stations of the individual modules of the modular biogas systems. The control electronics or the head-end stations generate control signals and collect data.

In one embodiment, a third housing contains at least one pump which, controlled via the central control and monitoring unit, manages the transport of the biomass within the respective modular biogas plant. Likewise, at least one heating device is provided in the third housing which, controlled by the central control and monitoring unit, maintains the temperature in the tanks of the modular biogas plant at least within a predetermined interval.

In one embodiment of the system according to the invention, parameters of the multiple modular biogas plants are compared in the central control and monitoring unit. Optimization criteria for the production of biomass in the individual modular biogas plants of the system can be obtained from the comparison. This has the advantage that the production process of biogas in the individual modular biogas plants can be optimized due to the statistics or the overview of several modular biogas plants through the central control and monitoring unit. This leads to a continuous improvement in the control of the process flows and consequently also to an optimized yield of biogas production from the respective biomass available in the production process.

The inventive system for the central monitoring and control of several modular biogas plants is characterized in that each of the modular biogas plants have a local data acquisition unit. According to another embodiment of the system, each module of the modular biogas plant can have its own (intelligent) head station. The intelligent head-end stations are also able to communicate with the central control and monitoring unit. The intelligent head stations, if available, take on smaller control tasks and safety functions. Each module has its own function profile (parameters etc.) stored in the associated head stations and can therefore operate almost independently.

Several of the modular biogas plants (regardless of their design) can be controlled, monitored and managed via the central control and monitoring unit. The advantage of central monitoring and control is that the software that controls the processes provides protection for the modular biogas plant, which ultimately leads to increased local security and availability of the biogas plants. Likewise one achieves thereby an optimization of the performance of the individual modular biogas plants, which can be achieved through a global benchmark and improvements in the logical control. In addition, the optimization of the performance of the individual modular biogas plants is also achieved through a trend analysis and, if necessary, corrective intervention on the part of the central control and monitoring unit. The central control and monitoring also leads to cost optimization and simplification with regard to predictive maintenance, the management of the local operator of the respective modular biogas plant and the scheduling with regard to repairs or replacement of components of the modular biogas plants.

Furthermore, the modular biogas plant according to the invention stands out by the fact that it has a simple construction, which saves time and money. All parts for the construction of a single modular biogas plant are already prefabricated and can be used immediately for assembly on the installation site. All necessary tools for setting up the biogas plant are also included in the scope of delivery. The construction and construction are accompanied on site by an expert.

### BRIEF DESCRIPTION OF THE DRAWINGS

In the following, exemplary embodiments are intended to explain the invention and its advantages in more detail with reference to the accompanying figures. The proportions in the figures do not always correspond to the real proportions, since some shapes are simplified and other shapes are shown enlarged in relation to other elements for better illustration, in which:

FIG. 1 is a schematic view of a single-stage biogas plant, according to the prior art;

FIG. 2 is a schematic view of a two-stage biogas plant, according to the prior art;

FIG. 3 is a plan view of the arrangement of the various modules of an embodiment of the modular biogas plant according to the invention;

FIG. 4 shows a plan view of another arrangement of the modules of the embodiment of the modular biogas plant according to FIG. 3;

FIG. 5 is a side view of a possible embodiment of a module, which is configures as a and is used in the modular biogas plant according to the invention;

FIG. 6 shows a front end view of the embodiment of the module according to FIG. 5;

FIG. 7 is a side view of a further embodiment of a module, which is configured as a tank and is used in the inventive modular biogas plant;

FIG. 8 shows a front end view of the module according to FIG. 7;

FIG. 9 shows a rear end view of the module according to FIG. 7;

FIG. 10 is a schematic view of the arrangement of the various modules according to an embodiment of the inventive modular biogas plant;

FIG. 11 is a schematic representation of an enclosure or housing, which contains at least one a pump and at least one heating device;

FIG. 12 shows a schematic representation of an embodiment of a housing which represents the gas storage;

FIG. 13 shows a schematic representation of an embodiment of a module which is used for feeding biomass to hydrolysis tanks;

FIG. 14 shows a schematic representation of an embodiment of a tank which is used in the inventive modular biogas plant;

FIG. 15 is a schematic representation of an embodiment of the inventive system, with which the individual modular biogas plants communicate with a central control and monitoring system;

FIG. 16 is a schematic representation of an embodiment of the inventive system, with which the individual modular biogas plants communicate with a central control and monitoring unit; and

FIG. 17 shows a schematic representation of the communication between the individual modular biogas plants with the cloud (according to the embodiment of FIG. 15), which is part of the inventive system.

### DETAILED DESCRIPTION OF EMBODIMENTS OF THE INVENTION

At the outset, it should be appreciated that like drawing numbers on different drawing views identify identical, or functionally similar, structural elements of the invention. It is to be understood that the invention as claimed is not limited to the disclosed aspects. Furthermore, for the sake of clarity, only reference symbols are shown in the individual figures which are necessary for the description of the respective figure.

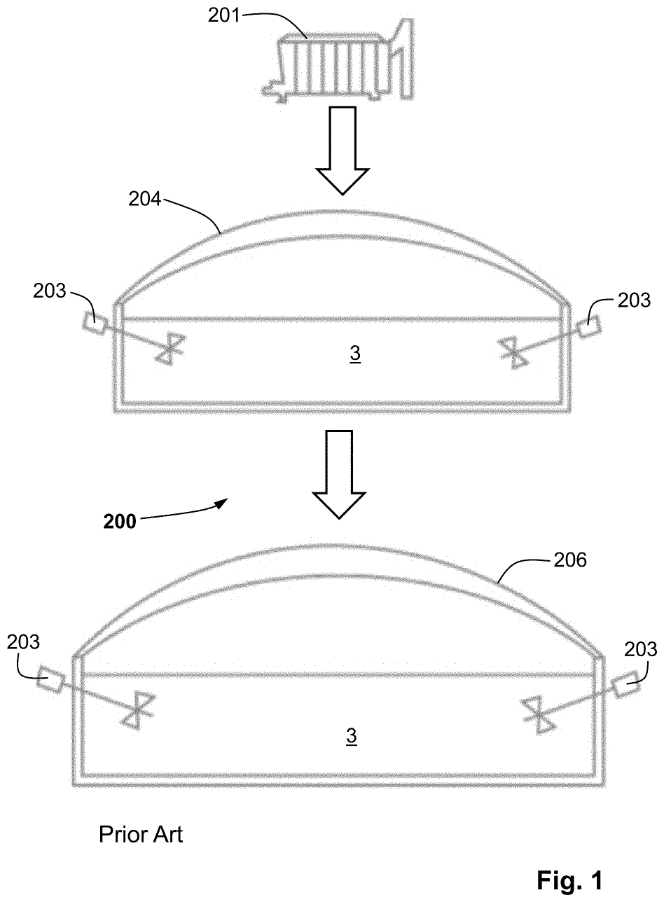

FIG. 1 shows a schematic representation of an embodiment of a single-stage biogas plant 200 according to the prior art. The biogas plant 200 comprises a conveying 201 with which biomass 3, which is to be processed in the biogas plant 200, is initially transported into a fermenter container 204. The biomass 3 can be agitated in the fermenter container 204 with at least one agitator 203, in order to improve the fermentation process in the fermenter container 204. From the fermenter container 204, the biomass 3 arrives in a fermentation residue storage 206. Here, the fermentation of the biomass 3 can be continued. The biomass 3 can also be moved here in the fermentation residue storage 206 with the at least one agitator 203.

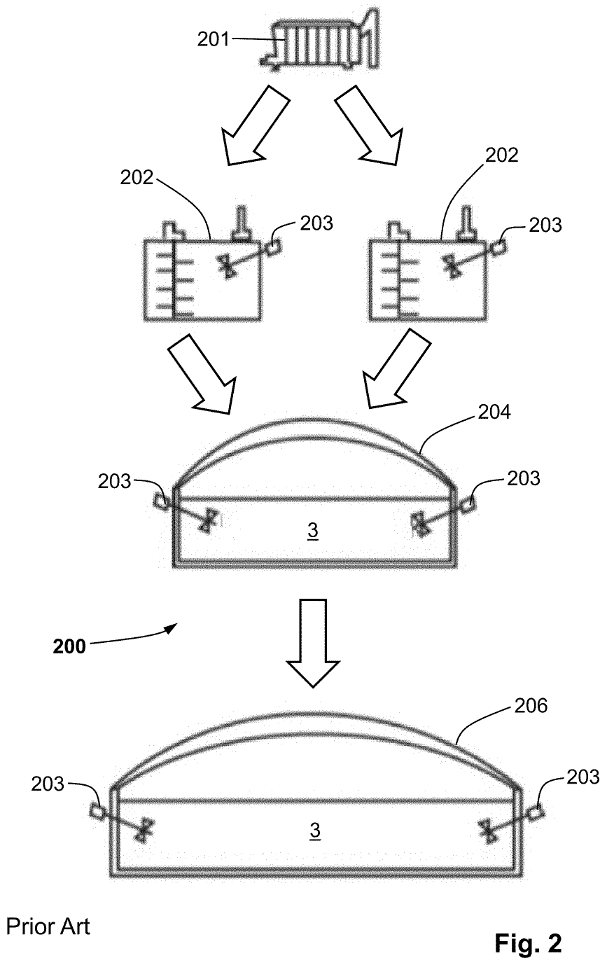

FIG. 2 shows a schematic view of an embodiment of a two-stage biogas plant 200 according to the prior art. In contrast to the single-stage biogas plant 200 (see FIG. 1), the two-stage biogas plant 200 is provided with two hydrolysis containers 202. With the conveyor 201, the biomass 3 is first transferred in the batch-method into one hydrolysis container 202 and then into the other hydrolysis container 202. The batch-method means that when the hydrolysis container 202 are filled alternately, for example, one hydrolysis container 202 is filled on one day and the other hydrolysis container 202 on the other day. Both hydrolysis containers 202 can also be provided with an agitator 203. The biomass 3 passes from the hydrolysis container 202 into the fermenter container 204. Agitators 203 are also provided here. After the end of the fermentation process or a decrease in the formation of biogas, the biomass 3 is transferred from the fermenter container 204 to the fermentation residue storage 206. Another production of biogas can possibly take place in the fermentation residue storage 206. The biomass 3 in the fermentation residue storage 206 can also be moved with agitators 203.

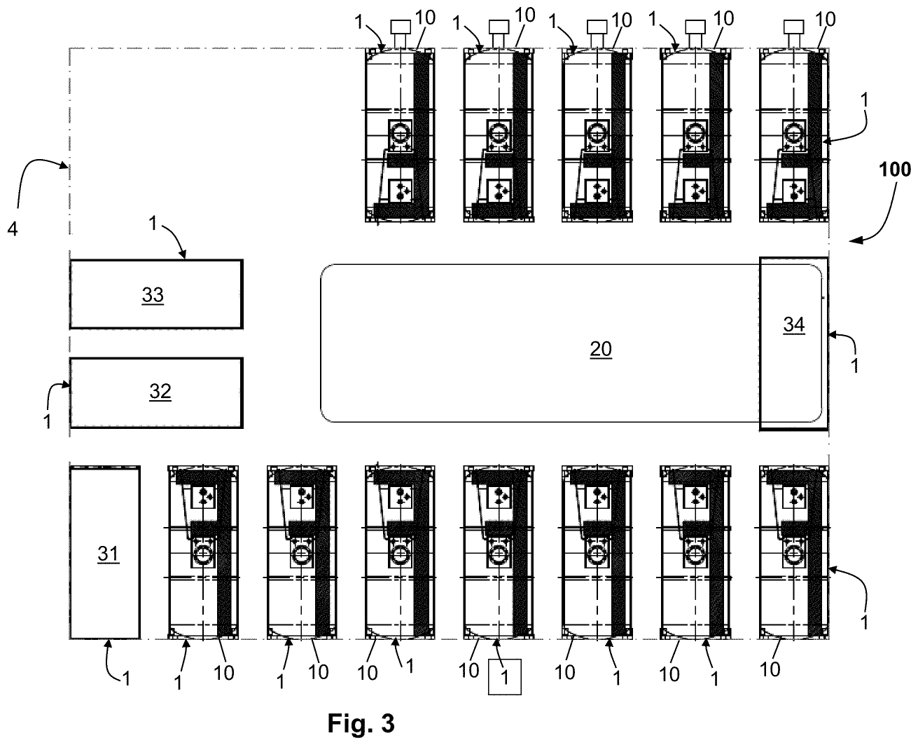

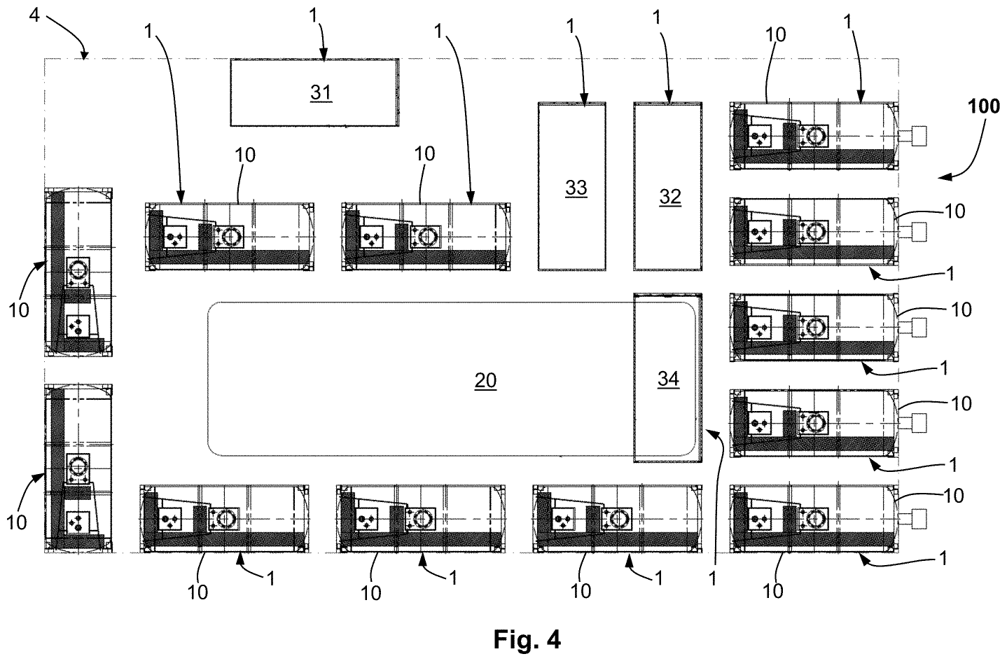

FIG. 3 and FIG. 4 show different embodiments of the setting of the inventive modular biogas plant 100. It can be seen from FIGS. 3 and 4 that the modular biogas plant 100 comprises a plurality of modules 1. The modules 1 are all of the same size. The same size is of particular advantage, as this makes the transport and production of the individual modules considerably easier and thus lowers costs. In addition, the fact that the modules 1 are equal in size enables them to be stacked or combined. Some of the modules 1 of the modular biogas plant 100 are designed as tanks 10. Another module 1 of the modular biogas plant 100 can be designed as a first housing 31. Likewise, a further module 1 can be designed as a second housing 32 and yet another module 1 can be designed as a third housing 33. In the housings 31, 32, 33 elements for controlling the modular biogas plant 100 as well as for generating energy from the biogas produced by the modular biogas plant 100 can be accommodated. Another module 1 of the modular biogas plant 100 is a transport housing 34. In the transport housing 34 a gas storage 20 can be accommodated for transport. For the operation of the modular biogas plant 100, the flexible gas storage 20 can be rolled out of the transport housing 34 and thus comes to rest on a portion of the footprint 4 of the modular biogas plant 100, as shown in FIGS. 3 and 4.

It goes without saying for a person skilled in the art that the embodiments of the modular biogas plant 100 shown in FIGS. 3 and 4 are only to be regarded as examples and therefore do not restrict the invention. It goes without saying for a person skilled in the art that the number and arrangement of the individual modules 1 can be changed with regard the performance of the modular biogas plant 100.

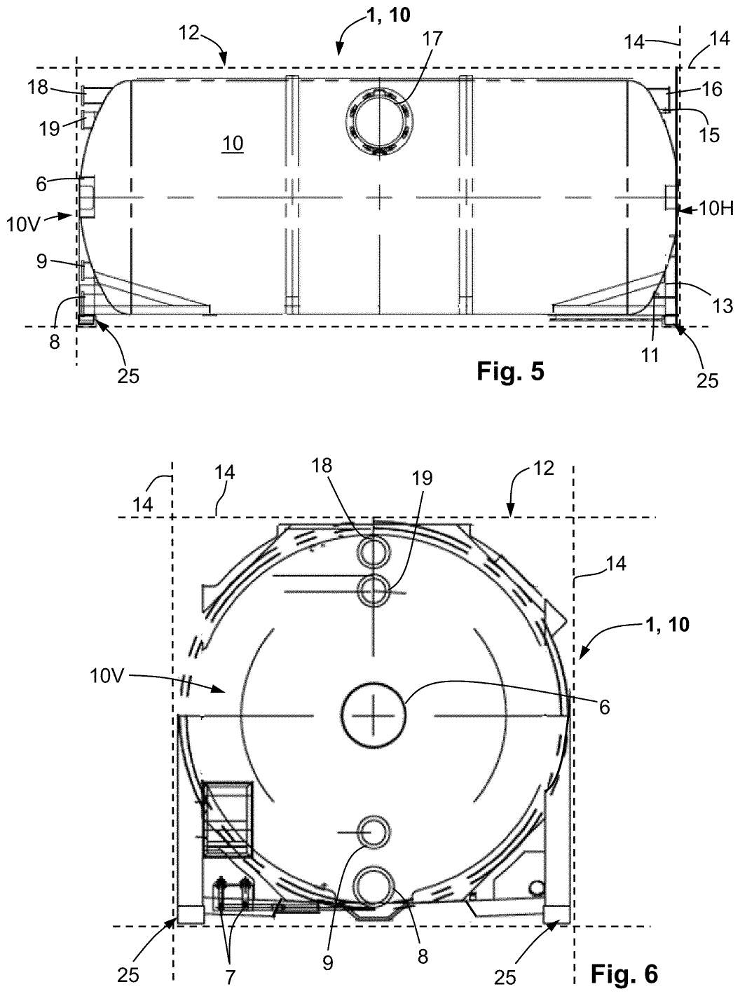

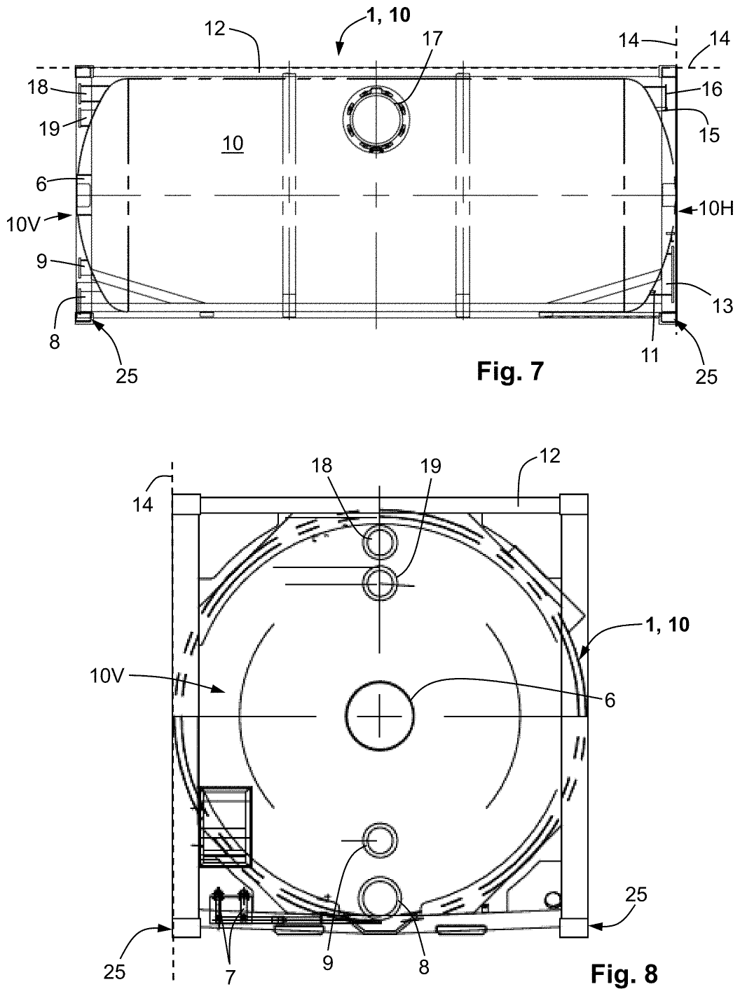

FIG. 5 shows a side view of a possible embodiment of a module 1, which is designed as a tank 10 and is used in the inventive modular biogas plant 100. In the embodiment of a tank 10 shown here, the latter has a plurality of positioning elements 25. The positioning elements 25 essentially serve to set up the tanks 10 in a stable and secure position on a surface (not shown) at an installation site provided for this purpose. The positioning elements 25 define a virtual, cuboid frame 12 which defines six side surfaces 14. The side surfaces 14 of the virtual, cuboid frame 12 form an envelope for the tank 10. All of the connections that are possible or required for the tank 10 and are adequately described in FIGS. 7 to 9 are located within the envelope.

FIG. 6 shows an end view of the embodiment of the module 1 (tank 10) according to FIG. 5. The positioning elements 25 define the virtual, cuboid frame 12, which has six side surfaces 14. The front view also shows that none of the connections of the tank 10 protrude beyond the envelope defined by the side surfaces 14.

FIG. 7 shows a side view of an embodiment of a module 1, which is a tank 10 for an embodiment of the inventive modular biogas plant 100. In the embodiment shown here, the positioning elements 25 for the tank 10 are attached to a rigid frame 12. The tank 10 is surrounded by the rigid frame 12. The frame 12 defines six side surfaces 14 which, as the virtual frame 12 of FIGS. 5 and 6, form an envelope for the tank 10. As can be seen from FIG. 5, no connections or add-on parts of the tank 10 extend beyond the side surfaces 14. This has the advantage that no prefabricated connections or add-on parts of the tank 10 can be damaged during transport. The rigid frame 12 for the tanks 10 is cuboid and has the same size as all other modules 1 of the modular biogas plant 100. In the embodiment shown here, the rigid frame 12 has lower positioning elements 25 and upper adjusting elements 26. As a result of the interaction of the upper positioning elements 26 of a lower module 1 with the adjusting elements 25 of an upper module 1, the modules 1 can be stacked securely.

As can be seen from FIG. 5, a manway 17 is provided in the upper region of the tank 10 on its side. The position of the manway 17 shown here is not mandatory. The manway 17 can be positioned as required. It goes without saying that the manway 17 is closed with a cover (not shown) during the operation of the modular biogas plant 100. At a front end 10V of the tank 10 a flange connection 18 for a gas pipe, a flange connection 19 for a pressure pipe, a flange connection 8 for a suction pipe and a flange connection 9 for gas injection are provided. The flange connections 8, 9, 18 and 19 described here can be equipped with the appropriate pipes (not shown) depending on the needs and function of the tank 10. The flange connections 8, 9, 18, 19 are pre-fabricated so that the assembly can be done quickly and easily during the set-up of the modular biogas plant 100. The embodiment shown here shows a possible arrangement of the connections. However, the invention is not limited to the number and arrangement of the connections shown here. Furthermore, a pipe section 6 for an agitator (not shown) can be provided at the front end 10V of the tank 10. If necessary, an agitator (not shown) can thus be inserted into the tank 10 at this point.

At the rear end 10H of the tank 10 an inspection glass 16 and a filling level probe 15 are provided. The maximum filling of the tank 10 can be censored via the filling level probe 15. Likewise, a flange connection 13 is provided for a feed screw (not shown), with which biomass 3 can be brought into the respective tank 10. A pressure sensor 11 is also provided. The position and number of the sensor system is only one example of many possibilities and is not to be understood as a limitation of the invention.

FIG. 8 shows a plan view of the front end 10V of the tank 10 as shown on FIG. 5. Here, too, it can be clearly seen that the side surfaces 14 of the rigid frame 12 represent an envelope for the tank 10. In addition to the flange connection 18 for the gas pipe, the flange connection 19 for the pressure pipe, the pipe section 6 for the agitator, the flange connection 9 for the gas injection and the flange connection 8 for the suction pipe a heating pipe 7 (with inlet flow and outlet flow) is provided. As already mentioned in the description of the other drawings, the arrangement of the connections described here is merely an example and is not to be regarded as a limitation of the invention. Via the heating pipe 7 the interior of the tank 10 or the biomass 3 located therein can thus be brought to the required temperature interval for the respective process.

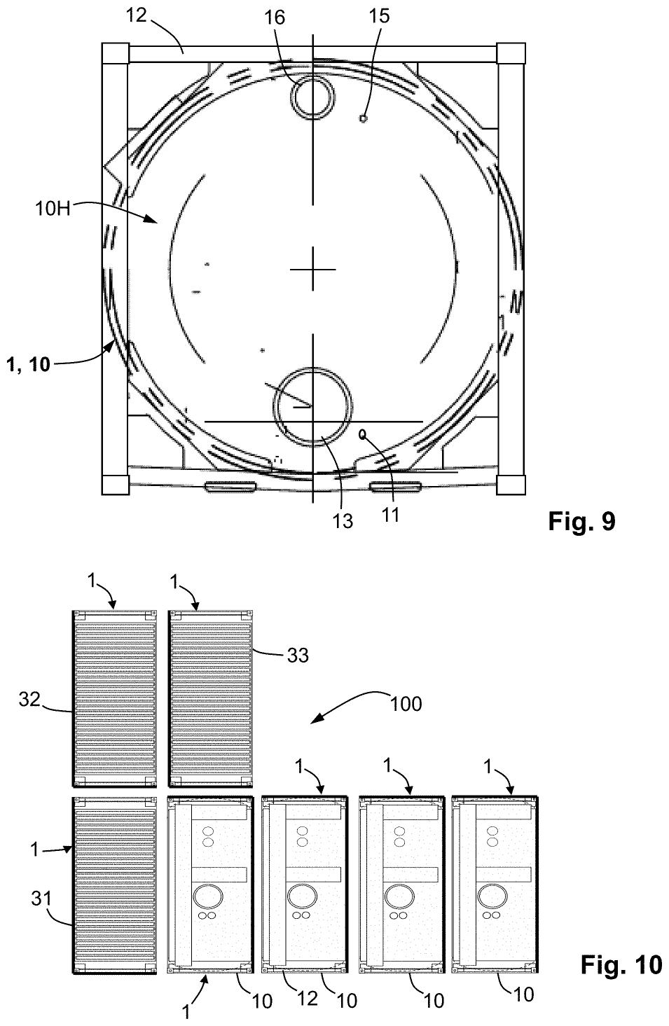

FIG. 9 shows a plan view of the rear end 10H of the tank 10. Here the inspection glass 16, the filling level probe 15, the flange connection 13 for the screw and the pressure sensor 11 are visible.

The embodiment of a tank 10 for the modular biogas plant 100 described in FIGS. 5 to 9 should not be construed as a limitation of the invention. It goes without saying for a person skilled in the art that the tanks 10 with different connections for the inlet and outlet pipes and the sensors and probe heads, respectively, can be designed. The exemplary embodiment described in FIGS. 5 to 7 is only to be understood as an example and should not be interpreted as a restriction of the invention.

FIG. 10 shows a further possible embodiment of the set-up of a modular biogas plant 100. In the embodiment shown here, the modular biogas plant 100 is made up of seven modules 1. Four of the modules 1 are designed as tanks 10. Three of the modules 1 are closed housings 31, 32, 33, which are designed in the form of standard containers (ISO sea containers with standard dimensions). It goes without saying that the invention is not intended to be restricted to standard containers. As can also be seen from FIG. 10, that the modules 1 are all the same size. As already mentioned in the description above, each of the tanks 10 is accommodated in a cuboid frame 12 and has the same size the as that of the housings 31, 32 or 33. The embodiment of the modular biogas plant 100, shown here, is configured a power under 100 kWh, which should not be regarded as a limitation of the invention.

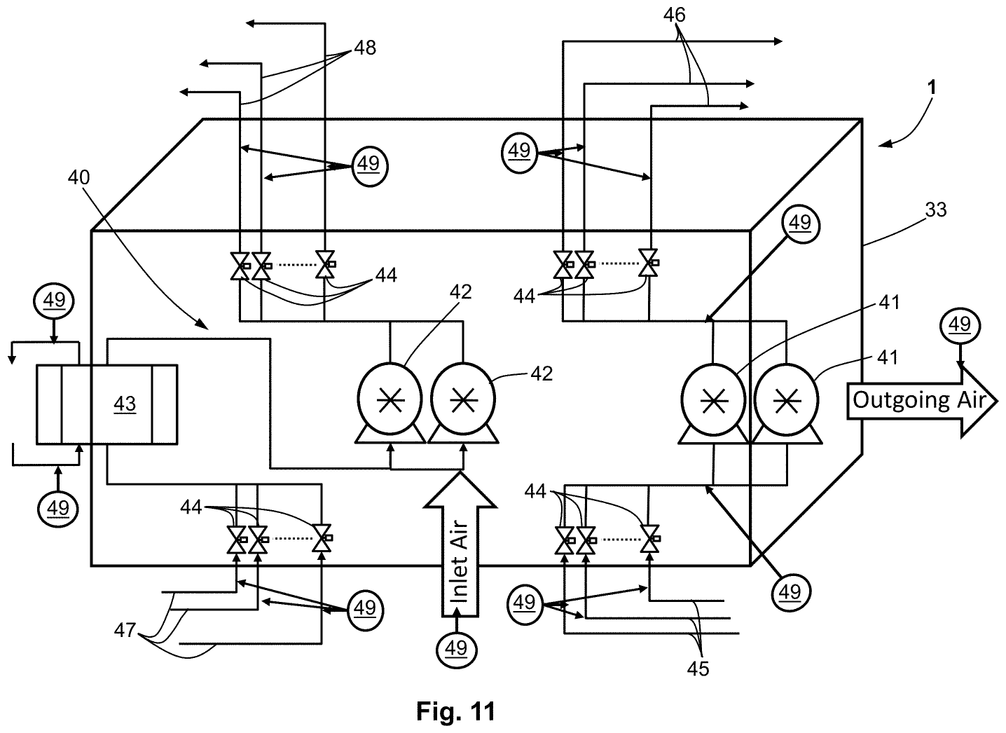

FIG. 11 shows a schematic representation of the internal structure of an embodiment of a module 1 of the modular biogas plant 100. The module 1 shown here is a third housing 33, has at least one pump 41 for the biomass 3 and at least one pump 42 for the cooling fluid/heating fluid of the heating device 40. In the embodiment shown here, two pumps 41 for the biomass 3 and two pumps 42 for the cooling fluid/heating fluid are provided. For this purpose, one of each of the pumps 41 or 42 is provided as a redundant pump, which steps in if the current pump 41 or 42 fails. Pipes 45 lead from the tanks 10 to the pump 41 for the biomass 3. After the pump 41 for the biomass 3, pipes 46 lead to the tanks 10. In each of the pipes 45 and 46 are provided with controllable valves 44. With the controllable valves 44 it is possible to fill or empty the tanks 10 of the modular biogas plant 100 in any desired manner. The heating device 40 is also accommodated in the third housing 33. The heating device 40 comprises a heat exchanger 43, which receives cooling water from the motors of the modular biogas plant 100 and sends the cooling water back to the motors. Via the heat exchanger 43, heating pipes 47 lead from the tanks 10 to the at least one pump 42 for the heating fluid. Heating pipes 48 lead from the pump 42 for the heating fluid to the tanks 10. In the heating pipes 47 from the tanks 10 and in the heating pipes 48 to the tanks 10, controllable valves 44 are provided. Through these controllable valves 44 the heating power can be distributed to the selected tanks 10 as required. A plurality of measuring points 49 are also provided for process automation in the third housing 33. The information from the measuring points 49 reach a central control and monitoring unit 120 which is provided in the second housing 32. Furthermore, the third housing 33 can be supplied with air for cooling the pumps 41 and 42, respectively. It is also possible to discharge exhaust air from the third housing 33. Corresponding measuring points 49 are also provided for the supply air and the exhaust air.

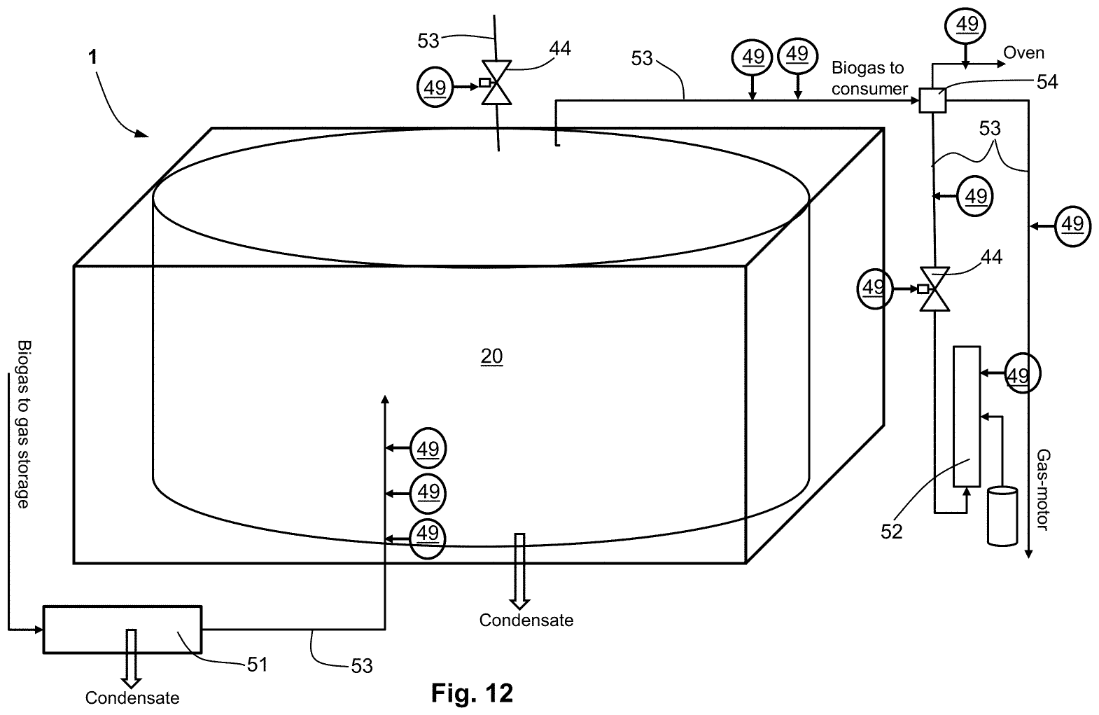

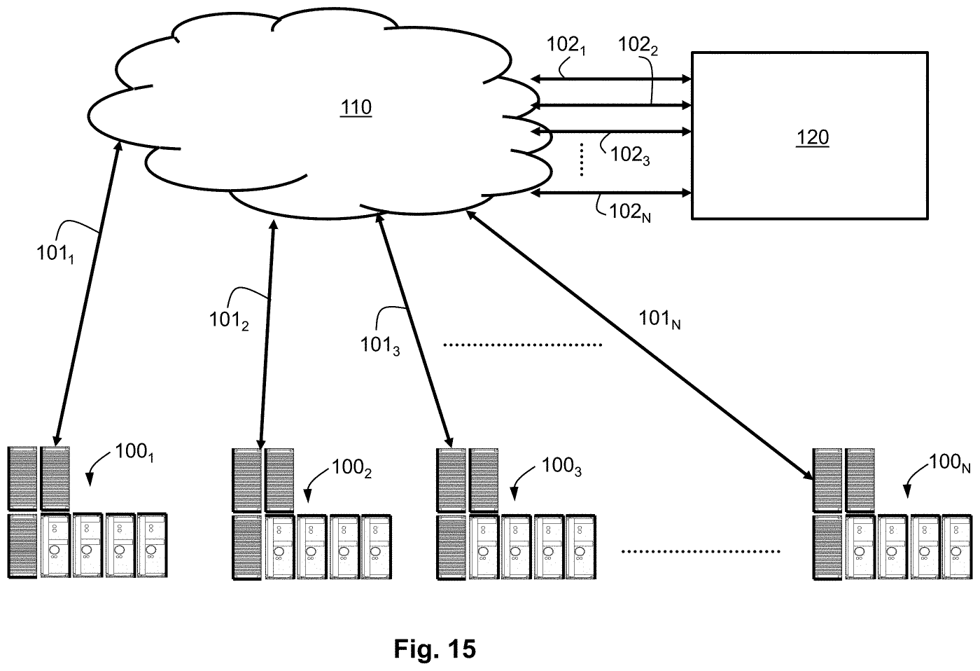

FIG. 12 shows a schematic representation of a possible embodiment of a further module 1 of the inventive modular biogas plant 100. The module 1 is the flexible gas storage unit 20. The biogas is transported to the gas storage unit 20 and from the gas storage unit 20 to the various consumers via a plurality of pipes 53. The transport of the biogas takes place without pressure or in a low pressure range. In the pipes 53 to the gas storage 20, the biogas is fed via a dehumidifier 51. The condensate from the dehumidifier 51 is collected and can be returned to the fermenter. Likewise, the condensate can also be removed from the gas reservoir 20. The pipe 53 from gas storage 20 leads the biogas to consumers. At a switch 54, the biogas can be directed to various consumers, such as, but without limitation of the invention, an oven for cooking or the gas engine (block-type thermal power plant), a gas torch 52 or another consumer (not shown here, like an oven, stove, boiler, burner or heater, etc.). Controllable valves 44 are provided in the pipe 53 for the excess pressure and in the pipe 53 to the gas flare 52. Likewise, a plurality of measuring points 49 are assigned to the pipes 53 and the controllable valves 44. With the corresponding measuring points 49, for example, the supply of the amount of biogas into the gas storage 20 can be determined. The corresponding measuring points 49, the consumption of biogas at the gas flare 52 can be determined. In addition, the amount of biogas that goes to the gas engine or the amount of biogas that is fed to a consumer, such as a burner or an oven for cooking, is determined with the measuring points 49. A corresponding intervention from a central control and monitoring unit 120 (see FIGS. 15 to 17) in the gas recycling is thus possible and, in parallel, also enables a bookkeeping of the flows of the biogas.

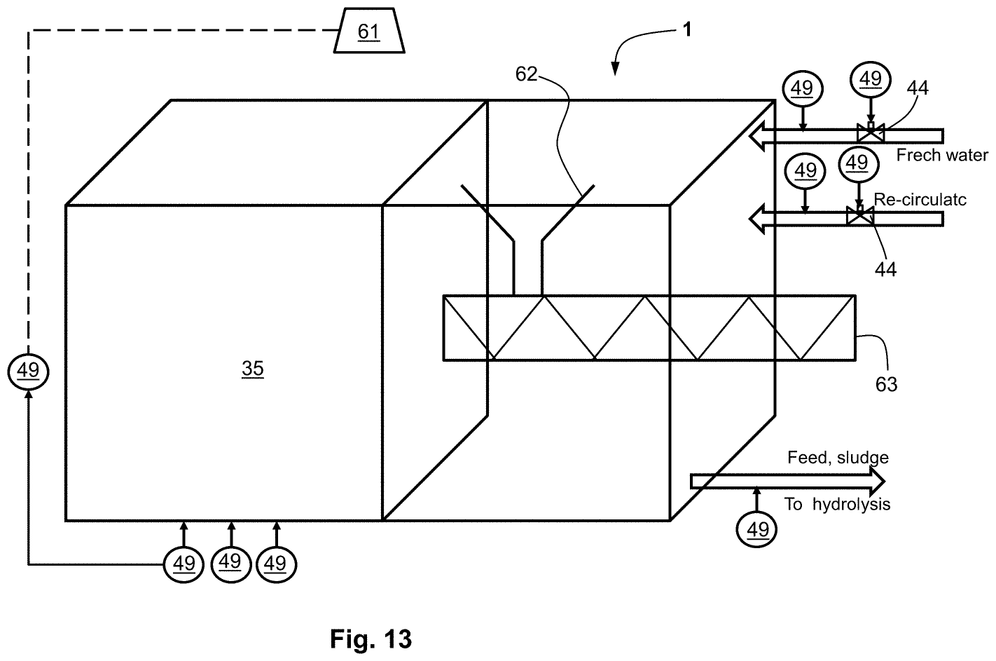

FIG. 13 shows a schematic illustration of an embodiment of a further module 1 that can be part of the modular biogas plant 100. The module 1 is a feeder 35 here. The feeder 35 comprises a funnel 62 into which the biomass that is to be fed to the modular biogas plant 100 is filled. Via a worm 63 the biomass is reduced to small pieces and fed to the feeder 35 together with water, so that a certain type of sludge is formed, which represents a pumpable mass. The feeder 35 (also called feeding module) is supplied with the feed materials and the recirculate together with water, process water or rainwater. As a result, a pumpable substrate mixture is generated, which is then fed to the process in the modular biogas plant 100. The supply of fresh water, industrial water or rainwater and the supply of recirculate is regulated with corresponding control valves 44. A webcam 61 can be assigned to the feeder 35, which can optionally be equipped with image recognition software in order to automatically recognize feed materials. Via the webcam 61, it is thus possible to see from the central control and monitoring unit 120 (see FIGS. 15 to 17) which biomass is entering the feeder 35. The amount of biomass fed to the feeder 35 can be recorded by appropriate sensors. This is also used for supervision and can thereby possibly avoid malfunctions in the modular biogas plant 100. Several measuring points 49 for process automation are also assigned to the feeder 35, to control the supply of fresh water and the supply of recirculate, as well as the removal of sludge for hydrolysis. These measuring points 49 enable a controlled and trouble-free operation of the feeder 35 and thus also of the entire modular biogas plant 100.

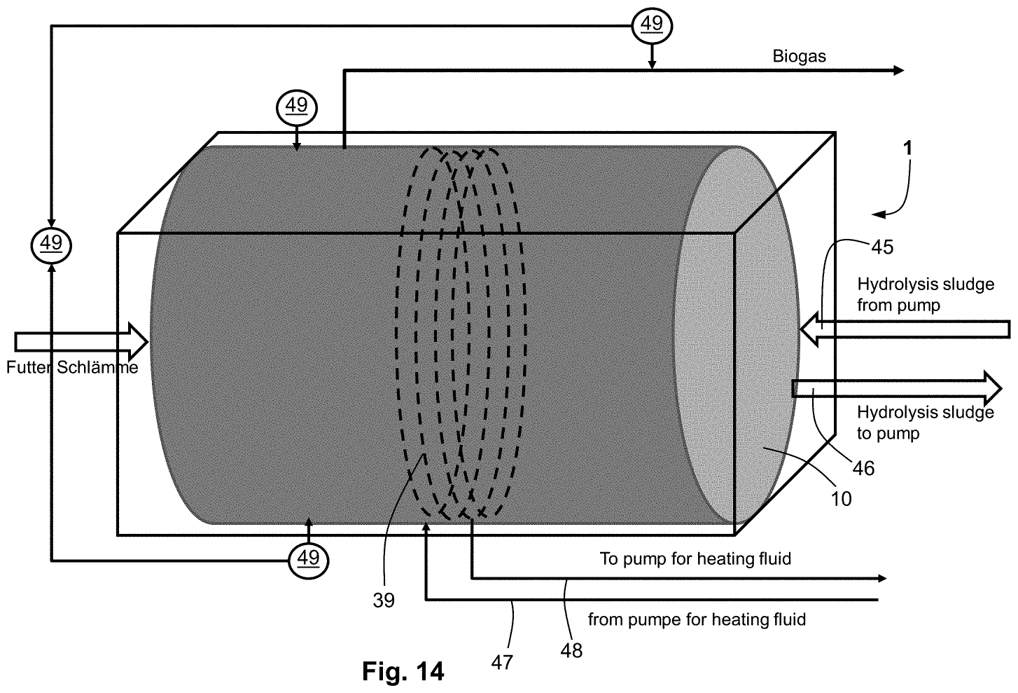

FIG. 14 shows a schematic representation of an embodiment of a further module 1 of the modular biogas plant 100. The module 1 shown here is a tank 10. A heater 39 for the tank 10 is connected to a heating pipe 47 to the tank 10 and to a heating pipe 48 from the tank 10. The heating pipes 47 and 48 are communicatively connected to the pump 42 (see FIG. 11) for the heating fluid. Generally, there are heating pipes 47 and 48 and the heat exchanger (cooling fluid/heating fluid) are defined for each module 1 and connected to the heating system with pump 42 and the heating control with a controllable valve and temperature sensors (both not shown). Feed sludge can be fed to the tank 10 for the production of biogas. Furthermore, the tank 10 is connected with a pipe 45 to the tank 10 and with a pipe 46 from the tank 10. Via pipe 45 and 46, hydrolysis sludge can be supplied to or removed from the tank 10 by means of the pump 41. The biogas formed in the tank 10 can be withdrawn without pressure or with low pressure and supplied to the gas storage 20 (not shown here) are. A plurality of measuring points 49 are also provided here, which monitor the transport of the sludge, the heating fluid, the biogas, etc. and report accordingly.

FIG. 15 shows schematically the communicative connection of several modular biogas plants 100₁, 100₂, . . . , 100_{N} with a central control and monitoring unit 120. Each of the modular biogas plants 100₁, 100₂, . . . , 100_{N} communicates via assigned communication links 101₁, 101₂, . . . , 101_{N} with a cloud 110. The cloud 110 communicates via communication links 102₁, 102₂, . . . , 102_{N} with the central control and monitoring unit 120. The communication links 102₁, 102₂, . . . , 102_{N} between the cloud 110 and the central control and monitoring unit 120 are assigned to the individual modular biogas plants 100₁, 100₂, . . . , 100_{N} to be monitored. The control signals, commands, warnings, etc. generated by the central control and monitoring unit 120 can be sent from the cloud 110 via the communication links 101₁, 101₂, . . . , 101_{N} to the individual modular plants 100₁, 100₂, . . . , 100_{N}. The central control and monitoring unit 120 is also provided so that the individual modular biogas plants 100₁, 100₂, . . . , 100_{N} are operated individually and automatically form the central control and monitoring unit 120. The schematic illustration in FIG. 15 shows a type of modular biogas plants 100₁, 100₂, . . . , 100_{N}. which are connected to the central control and communication unit 120 via the cloud 110. This is not to be construed as a limitation of the invention. It goes without saying for a person skilled in the art that different types and embodiments of the modular biogas plants 100₁, 100₂, . . . , 100_{N} can be operated and monitored via the central control and monitoring unit 120. If the communication with the superordinate central control and monitoring unit 120 does not come about, the local control 103 can also continue to operate the respective modular biogas plant 100₁, 100₂, . . . , 100_{N}. After a period to be defined, operators and responsible persons are informed via SCADA, visualization and as well as means of communication that there is a problem with the communication to the higher-level central control and monitoring unit 120.

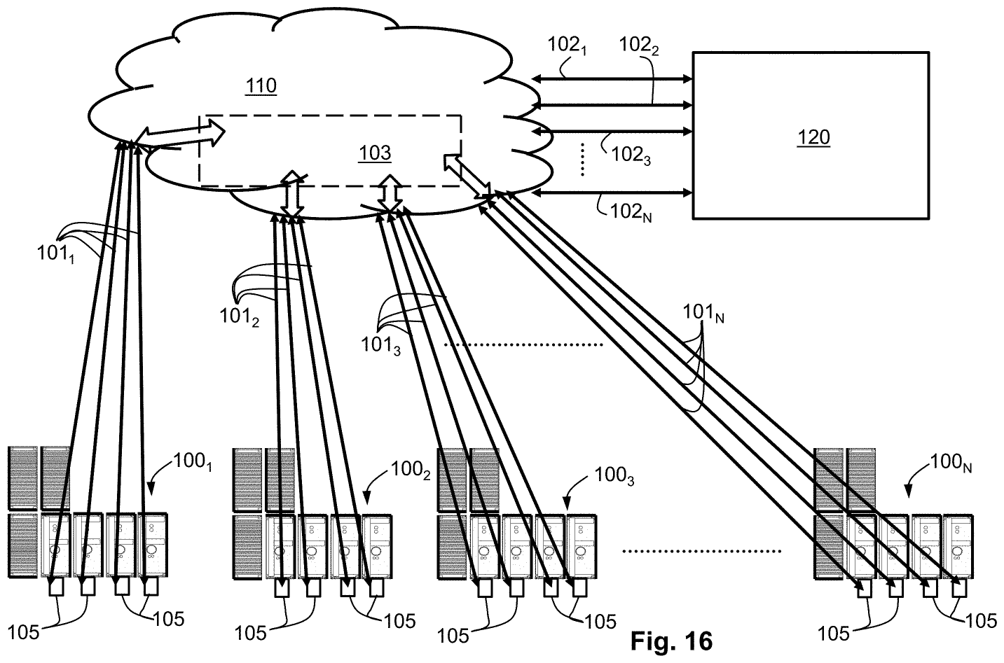

FIG. 16 shows a schematic representation of a further embodiment of the inventive system, how the individual modular biogas plants 100₁, 100₂, . . . , 100_{N} communicate with a central control and monitoring unit 120. In this embodiment, each of the modules 1 is provided with an intelligent head station 105. Each intelligent head station 105 can acquire data from the respective module 1, can at least partially control the respective module 1 and is communicatively connected to the cloud 110. The controller 103 for the modules 1 of the modular biogas plants 100₁, 100₂, . . . , 100_{N} is implemented in the cloud 110. The control signals, commands, warnings, etc. generated by the central control and monitoring unit 120 can be sent from the cloud 110 and the controller 103 to the individual intelligent head-end stations 105 of the individual modules 1 via the communication links 101₁, 101₂, . . . , 101_{N} of the modular biogas plants 100₁, 100₂, . . . , 100_{N}. The central control and monitoring unit 120 is also provided so that the individual modular biogas plants 100₁, 100₂, . . . , 100_{N} are operated individually and automatically by the central control and monitoring unit 120. The representation of the modular biogas plants 100₁, 100₂, . . . , 100_{N} with four tanks 10 is only used for description and is not to be interpreted as a limitation of the invention. According to the invention, a plurality of biogas plants 100₁, 100₂, . . . , 100_{N} can be managed by the central control and monitoring unit 120.

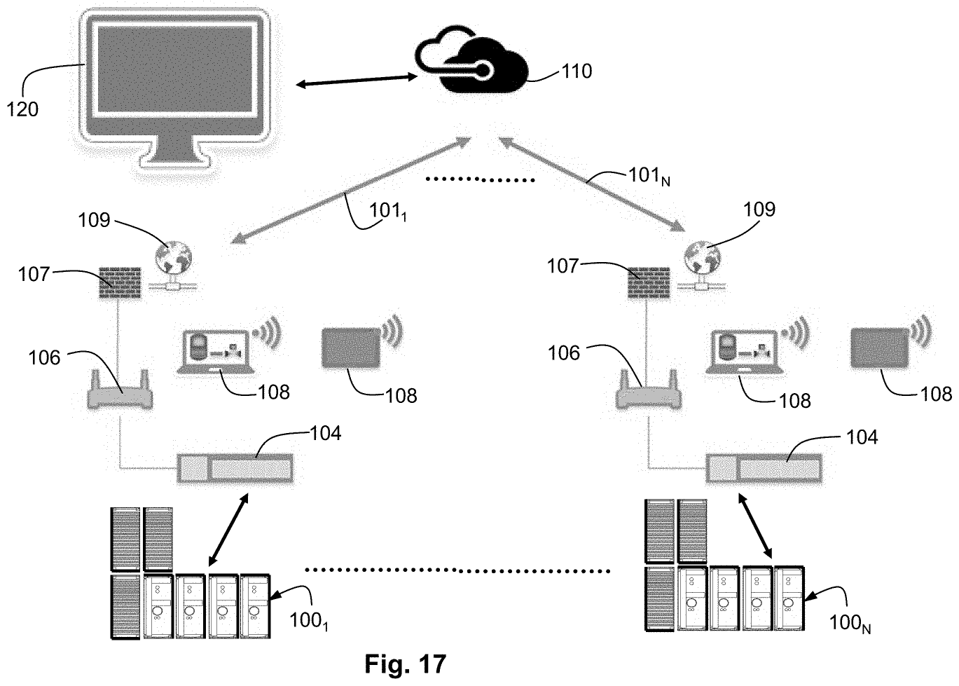

FIG. 17 shows a schematic representation of the communication between the individual modular biogas plants 100₁, 100₂, . . . , 100_{N} with the cloud 110 and the central control and monitoring unit 120. Each of the modular biogas plants 100₁, 100₂, . . . , 100_{N} supplies data and parameters to the respective local control and data acquisition unit 104. A respective communication device 106 is connected to the respective control and data acquisition unit 104. The respective communication device 106 communicates with the cloud 110 via a firewall 107 and the internet 109. The cloud 110 itself then communicates with the central control and monitoring unit 120. Instructions, commands, messages, etc. arrive from the central control and monitoring unit 120 via the cloud 110 and the Internet 109 and the firewall 107 at the modular biogas plants 100₁, 100₂, . . . , 100_{N}. Likewise, each of the modular biogas plants 100₁, 100₂, . . . , 100_{N} has at least one user interface 108 assigned. The user interfaces 108 can for example receive via a W-LAN messages and/or warnings generated from the central control and monitoring unit 120. With the user interface 108 the messages and/or warnings can be made available or displayed to the operator of the local modular biogas plants 100₁, 100₂, . . . , 100_{N}. The operator is thus informed centrally whether an error occurs in the respective local biogas plant 100₁, 100₂, . . . , 100_{N}. which, for example, requires current intervention by the operator himself. It is also possible for the operator to know and be informed in advance about any upcoming repairs or the replacement of components of the modular biogas plant 100₁, 100₂, . . . , 100_{N}.

The invention has been described with reference to preferred embodiments. It goes without saying for a person skilled in the art that changes and modifications can be made without departing from the scope of protection of the following claims.

### REFERENCE NUMBERS

1 Module
2 Biomass
3 Footprint
6 Pipe section
7 Heating pipe (inlet flow and outlet flow)
8 Flange connection for suction pipe
9 Flange connection for gas injection
10 Tank
10H Rear end of the tank
10V Front end of the tank
11 Pressure sensor
12 Virtual cuboid frame, rigid frame
13 Flange connection for feed screw
14 Side surface
15 Filling level probe
16 Inspection glass
17 Manway
18 Flange connection for gas pipe
19 Flange connection for pressure pipe
20 Gas storage
25 Positioning element
26 Upper positioning element
31 First housing
32 Second housing
33 Third housing
34 Transport housing
35 Feeder
39 Heater
40 Heating device
41 Pump
42 Pump
43 Heat exchanger
44 Controllable valve
45 Pipe
46 Pipe
47 Heating pipe from the tanks
48 Heating pipe to the tanks
49 Measuring point
51 Dehumidifier
52 Gas torch
53 Pipe
54 Switch
61 Webcam
62 Funnel
63 Worm
100 Modular biogas plant
100₁, 10O₂, . . . , 100_{N} Modular biogas plant
101₁, 101₂, . . . , 101_{N} Communication link
102₁, 102₂, . . . , 102_{N} Communication link
103 Control
104 Data acquisition unit
105 Intelligent head-end station
106 Communication device
107 Firewall
108 User Interface
109 Internet
110 Cloud
120 Central control and monitoring unit
200 Biogas plant
201 Conveyor
202 Hydrolysis container
203 Agitator
204 Fermenter container
206 Fermentation residue storage

What is claimed is:

1. A modular biogas plant comprising:
a plurality of modules;
a plurality of tanks, for receiving biomass, define a portion of the plurality of modules, wherein the tanks of the modular biogas plant comprise at least two hydrolysis tanks and at least one fermenter tank and being fluidly connected to one another;
at least one gas storage for the biogas generated in the at least one fermenter tank of the modular biogas plant; and
a plurality of positioning elements is at least provided to the tanks, wherein the positioning elements define six side surfaces which form an envelope for each tank.

2. The modular biogas plant as claimed in claim 1, wherein the plurality of positioning elements is attached to a rigid cuboid frame, which defines the six side surfaces and the side surfaces of the rigid, cuboid frame forms an envelope for the tank.

3. The modular biogas plant as claimed in claim 1, wherein the modules of the modular biogas plant further comprise at least two closable housings, wherein each of the closable housings has the size of the rectangular frame, and wherein each of the closable housings is designed to be accessible on at least one side surface and the remaining side surfaces of the frame are provided with a cladding.

4. The modular biogas plant as claimed in claim 3 comprising:
a first housing which contains a block-type thermal power plant that uses the biogas generated in the modular biogas system as an energy source, and
a second housing which has control electronics for the entire modular biogas plant, at least one pump which manages the controlled transport of the biomass within the modular biogas plant, and at least one heating device, which can be connected to the tanks for controlled temperature management within the tanks, wherein the at least one pump is connected to the tanks via a pipe system and a controllable and regulatable valve is assigned to each of the tanks, and wherein the heating device has a pipe system which leads to the tanks, each tank being assigned a controllable and regulatable valve in order to set a required temperature range of the biomass in the tanks.

5. The modular biogas plant as claimed in claim 3 comprising:
a first housing containing a combined heat and power unit that uses the biogas generated in the modular biogas plant as an energy source,
a second housing that contains control electronics for the entire modular biogas plant, and
a third housing, the at least one pump, which brings about the transport of the biomass within the modular biogas plant, and at least one heating device, which is used for controlled temperature management within the tanks, wherein the at least one pump is connected to the tanks via a pipe system and a controllable and regulatable valve is assigned to each of the tanks, and wherein the heating device has a pipe system which leads to the tanks, each tank being assigned a controllable and regulatable valve in order to set a required temperature range of the biomass in the tanks.

6. The modular biogas plant as claimed in claim 1, wherein each of the tanks has a connection for the supply and discharge of biomass, a connection for a supply of biogas and a connection for a discharge of biogas.

7. The modular biogas plant as claimed in claim 1, wherein a fermentation residue storage is provided, which receives fermented residues from the tanks, which are fermenter tanks, of the modular biogas plant.

8. The modular biogas plant as claimed in claim 1, wherein a pressure less gas storage is provided, which at least for receiving biogas from the modular biogas plant, for delivering biogas to the combined heat and power plant and for returning biogas in the tanks, which are fermentation tanks, is formed.

9. The modular biogas plant as claimed in claim 8, wherein the fermentation residue storage is connected to the gas storage for supplying biogas from the fermentation residue storage into the gas storage.

10. The modular biogas plant as claimed in claim 9, wherein the pressure less gas storage is made of a flexible material and defines an end which is connected to a cladding of a transport housing of the gas storage.

11. A method for operating a modular biogas plant which comprises at least a plurality of tanks for receiving biomass, wherein at least two of the tanks are hydrolysis tanks and at least one tank is a fermenter tank and at least one gas storage for the biogas generated in the modular biogas plant, comprising the following steps:
batch-wise filling of the hydrolysis tanks with biomass, wherein a first temperature range and a first pH range are predominating in the hydrolysis tanks;
transferring the biomass from the at least one hydrolysis tank to the at least one fermenter tank by means of a pump;
producing in the at least one fermenter tank biogas from the biomass, transferred from the at least one hydrolysis tank to the at least one fermenter tank, wherein the formation takes place at a second temperature range and a second pH range in the at least one fermenter tank; and
monitoring continuously a production rate of the biogas and if the production rate of the biogas in the at least one of the fermenter tanks falls below a predefined value, biomass is supplied from one of the hydrolysis tanks until the production rate is again above the predefined value.

12. The method according to claim 11, wherein at least the hydrolysis tanks and the fermenter tanks have assigned controllable valves and the hydrolysis tanks and the fermenter tanks are connected via pipes to at least one pump, so that the hydrolysis tanks and/or the fermenter tanks can be connected in any combination such that biomass can optionally be supplied to the hydrolysis tanks and/or the fermenter tanks or optionally removed from them.

13. The method according to claim 11, wherein the hydrolysis tanks and the fermenter tanks are each provided with an inlet and outlet for heating fluid and a controllable valve is provided in each inlet and outlet, so that the hydrolysis tanks and/or fermenter tanks can be fed in a controlled manner with heating fluid.

14. The method according to claim 11, wherein at least one fermentation residue storage is provided, which has assigned a controllable valve in a pipe to the fermentation residue storage, so that biomass is supplied in a controlled manner from at least one of the fermenter tank to the fermentation residue storage.

15. A system for computer-aided, centralized monitoring and control comprising: several modular biogas plants, wherein each modular biogas plant has at least two hydrolysis tanks, at least one fermenter tank, at least one pressure less gas storage tank and several housings;
at least one data acquisition unit is assigned to several individual and movable modules of each of the modular biogas plants and each of the modules has at least one actuator and/or at least one sensor and/or at least one measuring point, which are communicatively connected to the least one data acquisition unit;
a communication device which is assigned to each of the modular biogas plants and delivers data from the data acquisition unit to a cloud or receives data from the cloud;
a central control and monitoring unit which is communicatively connected to the cloud in order to monitor the modular biogas plants in a centralized manner and to control them automatically; and
a user interface is assigned to each of the modular biogas plants to which messages or warnings can be transmitted from the central control and monitoring unit.

16. The system according to claim 15, wherein the data acquisition unit is communicatively connected to at least one controller and the controller is communicatively connected to the cloud.

17. The system according to claim 16, wherein each of the modules is provided with an intelligent head station and each intelligent head station records data of the respective module, at least partially controls the respective module and is communicatively connected with the cloud, and the controller for the modules is implemented in the cloud.

18. The system according to claim 15, wherein a first housing contains a block-type thermal power plant, which can be operated via the central control and monitoring unit in order to thus ensure that the biogas produced by the modular biogas systems be used as an energy source.

19. The System according to claim 15, wherein a third housing comprises at least one pump which, controlled via the central control and monitoring unit in connection with the controller, carries out the transport of the biomass within each of the modular biogas plants, and comprises at least one heating device which, controlled via the central control and monitoring unit in connection with the controller, holds the temperature in the tanks of each of the modular biogas plants at least within a specified interval.
